(19) United States
(12) Patent Application Publication
(10) Pub. No.: US 2026/0090761 A1
(43) Pub. Date: Apr. 2, 2026

(54) WEARABLE MEASURING DEVICE, ASSISTANCE SYSTEM COMPRISING A WEARABLE MEASURING DEVICE, METHOD FOR ASCERTAINING A MUSCLE HARDNESS OF A SKELETAL MUSCLE

(71) Applicant: J.Schmalz GmbH, Glatten (DE)

(72) Inventor: Raphael SINGER, Horb am Neck (DE)

(73) Assignee: J.Schmalz GmbH, Glatten (DE)

(21) Appl. No.: 18/880,032

(22) PCT Filed: Jul. 4, 2024
(86) PCT No.: PCT/EP2024/068945

§ 371 Date: Dec. 30, 2024

(30) Foreign Application Priority Data
Jul. 27, 2023 (DE) 10 2023 119 950.1

(51) Int. Cl. A61B 5/00

(52) U.S. Cl. CPC A61B 5/4519; A61B 5/6802

(57) ABSTRACT

A wearable measuring device comprising a measuring apparatus that comprises at least two measuring units for monitoring a skeletal muscle of a person (or animal, etc.), and comprising a holding apparatus for attaching the measuring apparatus to a body part of the person having the skeletal muscle. It is provided that the measuring units each comprise a force sensor and an associated spring unit, the spring units differing from one another in their spring constant, that the measuring units can be attached to the body part in such a way that the spring units can be tensioned by a particular different section of the skeletal muscle, and that the force sensors are designed to detect a tensioning force of the associated spring unit.

The present invention relates to a wearable measuring device comprising a measuring apparatus having at least two measuring units and a holding apparatus for attaching the measuring apparatus to a body part of a person. Furthermore, the present invention relates to an assistance system comprising such a measuring device and a wearable exoskeleton. Furthermore, the present invention relates to a method for ascertaining a muscle hardness of a skeletal muscle of a person.

Wearable exoskeletons can be used to transfer loads placed on a user, for example when lifting heavy objects, either directly into the ground or at least through selected areas of the user's body. In this way, the user's joint and muscle system can be protected.

In order for an exoskeleton to effectively relieve a user's load, it is necessary to determine the user's assistance needs. For example, it is known to monitor a skeletal muscle of the user and derive the need for assistance from this. Traditionally, wearable measuring devices are used to monitor a skeletal muscle. Such measuring devices comprise a measuring apparatus for monitoring the skeletal muscle and a holding apparatus for attaching the measuring apparatus to a body part having the skeletal muscle. In this regard, publications WO 2018 050 191 A1 and ES 2 335 337 B1, for example, disclose wearable measuring devices in the form of force sensor bracelets. Publications CN 111 150 373 B and KR 101 675 577 B1 disclose wearable measuring devices in the form of gas-pressure sensor bracelets.

However, known force sensor bracelets and gas-pressure sensor bracelets have the disadvantage that the measurement result is distorted by various effects. For example, the degree of tightness with which the bracelets are worn has an influence on the measurement result. In addition, the measurement result is also affected by bulges in the monitored skeletal muscle, which occur during movement and are accompanied by a change in circumference.

The object of the invention is to improve skeletal muscle monitoring in the case of a wearable measuring device.

This object is achieved according to the invention by a wearable measuring device having the features of claim 1.

The dependent claims and the description indicate advantageous variants and embodiments.

According to the invention, a wearable measuring device is provided. The measuring device comprises a measuring apparatus that comprises at least two measuring units for monitoring a skeletal muscle of a person. The measuring device also comprises a holding apparatus for applying the measuring apparatus to a body part of the person having the skeletal muscle. Preferably, the body part is an extremity of the person, in particular an arm or a leg.

It is now provided that the measuring units each comprise a force sensor and an associated spring unit, the spring units each having different spring constants from one another. The measuring units can be attached to the body part in such a way that the spring units can be subjected to force or tensioned by a particular different portion of the skeletal muscle, and that the force sensors are designed to detect a tensioning force of the associated spring unit.

The invention is based on the approach that a skeletal muscle can be viewed as a grid or an array of spring units having the same spring constant. The strength or value of the spring constant of the skeletal muscle varies with the degree of contraction of the skeletal muscle. If the measuring units according to the invention are attached to the body part in such a way that the spring units can be tensioned by in each case a different portion of the skeletal muscle, the spring units will each form with the relevant, associated portion of the skeletal muscle a spring arrangement with an equivalent spring constant. Because the spring constants of the spring units differ from each other, the equivalent spring constants of the resulting spring arrangements also differ from each other. If the monitored skeletal muscle is contracted, a greater tensioning force will be transferred to the spring unit having the higher spring constant than to the spring unit having the lower spring constant. On the basis of the tensioning forces detected by the force sensors, the muscle hardness of the skeletal muscle can then be ascertained or estimated as a spring constant. In the idealized case, in which the skeletal muscle has a linear spring characteristic curve, it would be possible to calculate the spring constant of the skeletal muscle. In the real case, due to various effects, an estimate is made instead, but with sufficient accuracy. This approach overcomes the disadvantages previously mentioned in connection with force sensor bracelets and gas-pressure sensor bracelets. Consequently, the user's assistance requirements can be derived from the spring constant of the skeletal muscle in a user-friendly and reliable manner. The ascertainment of the spring constant of the skeletal muscle is largely independent of influencing factors such as the person using the measuring device or the time or muscle contraction at which the measuring device is applied. With a constant level of muscle contraction, a comparable spring constant can always be ascertained regardless of such influencing factors.

Preferably, the spring constant of the one spring unit is at least 1.1 times the spring constant of the other spring unit. Designing the spring units in this way ensures that the sensor signals of the force sensors are sufficiently different from each other for an accurate ascertainment of the spring constant of the skeletal muscle.

Preferably, the spring units according to the invention each comprise only a single spring element. However, the spring units can also each comprise a plurality of spring elements that are connected, for example, in series or parallel to one another.

The force sensors according to the invention are designed to detect a tensioning force of the associated spring unit, i.e. a tensioning force of the spring unit of the same measuring unit. Depending on the design of the measuring units, the force sensors can measure the tensioning force as a pulling force or as a pressing force. In the context of the disclosure, the term “force” means not only a force in the strict sense but also a surface pressure and a gas pressure. Consequently, the force sensors according to the invention can also detect the tensioning force as a surface pressure or as a gas pressure, at least in some embodiments.

In particular, the measuring device comprises only a single measuring apparatus, so that only a single skeletal muscle of the person can be monitored as intended using the measuring device. However, the measuring device can also comprise a plurality of measuring apparatuses, each of which can then be assigned to a different skeletal muscle of the same body part of the person. This makes it possible to monitor a plurality of different skeletal muscles of the same body part simultaneously using the same measuring device. If the body part is, for example, an arm, a measuring device having two measuring apparatus can monitor both the biceps brachii muscle and the triceps brachii muscle simultaneously.

In some preferred embodiments, the measuring device comprises an evaluation unit that which is designed to ascertain a spring constant of the skeletal muscle as a function of sensor signals from the force sensors. For example, the evaluation unit ascertains the spring constant according to a look-up table, according to a characteristic map, according to a calculation rule or according to a calculation network. In some alternative embodiments, the measuring device itself is free of an evaluation unit. There can then be an evaluation unit external to the measuring device, which evaluation unit is connected to the force sensors via communication technology and is designed to ascertain the spring constant of the skeletal muscle as a function of the sensor signals from the force sensors.

Various embodiments are possible with regard to the design of the spring units. Preferably, at least one of the spring units comprises a metal spring or an elastomer spring. By using such springs, the measuring device can be realized cost-effectively.

With regard to the material of the spring units, materials that allow a linear or at least substantially linear spring characteristic curve are preferred. The spring units can, for example, be made of a textile material or a metal material. The spring constant of the spring units is preferably in the order of magnitude of the spring constant of the skeletal muscle to be monitored.

Various embodiments are also possible with regard to the shape of the spring units. Preferably, at least one of the spring units is cylindrical, spiral-shaped, plate-shaped, bar-shaped, band-shaped or strand-shaped. Such spring units are available at low cost. Due to the compact design, an overall compact measuring device can also be realized. This is accompanied by a high level of comfort for the user.

In some preferred embodiments, at least one of the spring units comprises a gas-pressure spring. Gas-pressure springs have the advantage that they have a spring characteristic curve having an almost linear progression. A spring characteristic curve having a linear or almost linear progression makes possible a particularly precise ascertainment of the spring constant of the skeletal muscle.

Various embodiments are also possible as regards the type of force sensor. Preferably, at least one of the force sensors comprises at least one force-dependent electrical resistor, at least one force-dependent electrical capacitor, at least one force-dependent electrical inductor or at least one piezoelectric element. Force sensors having a force-dependent electrical resistor are also known as FSR sensors (force-sensing resistors) or DMS elements (strain gauges). Force-dependent electrical resistors, capacitors, inductors or piezoelectric elements are characterized by their low installation height, so that a particularly compact measuring device can be realized by using force-dependent electrical resistors, capacitors, inductors or piezoelectric elements.

In some preferred embodiments, it is provided that the spring units have a first end portion and a second end portion, the associated force sensor being operatively connected to the spring unit through the first end portion, and the spring unit being operatively connectable or operatively connected to the skeletal muscle through the second end portion. When the measuring device is arranged as intended, the spring units are located between the skeletal muscle and the force sensors. This arrangement of the force sensors has the advantage that a communication connection of the force sensors is facilitated, in particular a communication connection to the evaluation unit mentioned above.

In some preferred embodiments, it is provided that the spring units have a first end portion and a second end portion, the associated force sensor being operatively connected to the spring unit through the first end portion, and the spring unit being operatively connectable or operatively connected to the skeletal muscle through the force sensor. When the measuring device is arranged as intended, the force sensors are located between the skeletal muscle and the spring units. Even with such an arrangement of the spring units, the spring units can be tensioned by the skeletal muscle.

In some preferred embodiments, the measuring apparatus comprises at least three measuring units. By increasing the number of measurement units, the robustness and/or precision in ascertaining the spring constant of the skeletal muscle can be increased. In particular, the spring units of the measuring units each have a different spring constant. Alternatively, the spring units of at least two measuring units have the same spring constant. At least one of the measuring units is therefore redundant and can be used, for example, to check the plausibility of the sensor signals of the other measuring units.

In some preferred embodiments, the holding apparatus comprises a holding strap, a holding cuff or a holding bandage. Such holding apparatuses make possible a safe and precise attachment of the measuring apparatus to the body part. In addition, such holding apparatuses are associated with a high level of comfort for the user.

In some preferred embodiments, it is provided that the holding apparatus comprises a support structure, in particular a rigid support structure, the measuring units being arranged on the support structure. The arrangement of the measuring units on the common support structure facilitates the proper attachment of the measuring units to the body part. Particularly preferably, the support structure is attached to the holding strap, the holding cuff or the holding bandage in an in particular detachable manner.

In some preferred embodiments, it is provided that the spring units extend radially in relation to the body part when the measuring device is arranged as intended. With such an alignment of the spring units, the spring units are subjected by the skeletal muscle to a tensioning force that acts in the longitudinal extension of the spring units. In particular, in this embodiment the measuring units are arranged next to one another on the aforementioned support structure, the spring units extending away from the support structure in the same direction.

In some preferred embodiments, it is provided that the spring units extend in the circumferential direction of the body part along the skeletal muscle when the measuring device is arranged as intended. The two ends of the same spring unit are preferably arranged one behind the other in the circumferential direction. Such a design of the measuring device also makes possible a sufficiently accurate ascertainment or estimation of the spring constant of the skeletal muscle. Preferably, the spring units are arranged such that, when the measuring device is arranged as intended, they are axially offset from one another in relation to the longitudinal center axis of the body part. In order to detect the tensioning force of the spring units, the force sensors can each be operatively connected to one of the ends of the associated spring unit. Particularly preferably, the spring units in this embodiment are designed as elastic bands, in particular made of textile.

The object to be achieved is also achieved by an assistance system having the features of claim 14. The assistance system comprises a wearable exoskeleton having at least one controllable actuator. In addition, the assistance system comprises a measuring device designed as described above. The assistance system further comprises a control device that is designed to control the actuator of the exoskeleton as a function of information that can be provided by the measuring device. Said information comprises, in particular, a spring constant of a skeletal muscle, ascertained or estimated by the measuring device, to which the measuring device is assigned.

With regard to the advantages that can be achieved with the method, reference is made to the statements in this respect that relate to the assistance system. The features described in connection with the measuring device can serve for the further development of the assistance system.

It should be noted that the measuring device is not limited to use in an assistance system having an exoskeleton. The measuring device can also be used, for example, as part of a digital ergonomics assessment device or in training to track changes in muscle fitness.

The object to be achieved is also achieved by a method having the features of claim 15, which serves to ascertain a muscle hardness of a skeletal muscle of a person. It is provided that at least two spring units, which differ from one another in their spring constant, are attached to a body part of the person having the skeletal muscle in such a way that the spring units can be tensioned by a particular different portion of the skeletal muscle, that the tensioning forces of the spring units are detected in particular continuously, and that a spring constant of the skeletal muscle is ascertained as muscle hardness as a function of the detected tensioning forces.

Preferably, the method is carried out by a wearable measuring device designed as described above.

With regard to the advantages that can be achieved with the method, reference is made to the statements in this respect that relate to the measuring device. The features described in connection with the measuring device can serve for the further development of the method.

The invention is described in more detail below with reference to the figures, the same or functionally equivalent elements possibly being provided with reference signs only once. The description serves as an example and is not to be understood as limiting. In the figures:

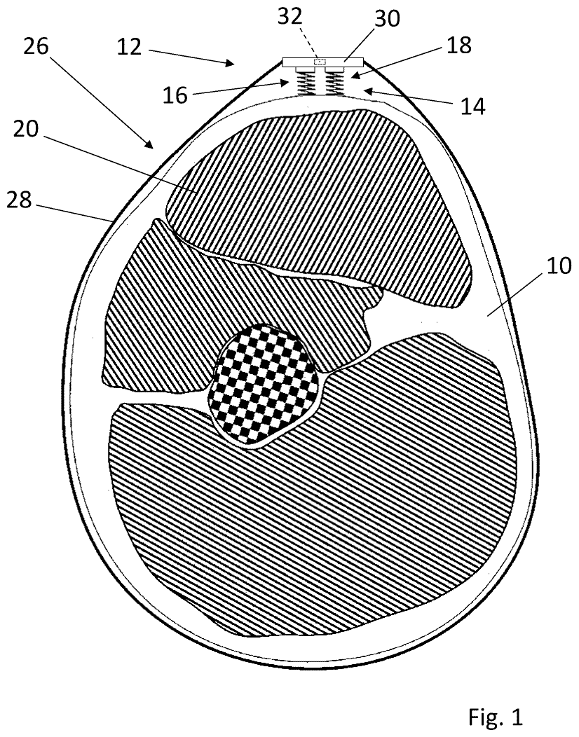

FIG. 1 shows a sectional view of a body part with a measuring device attached thereto,

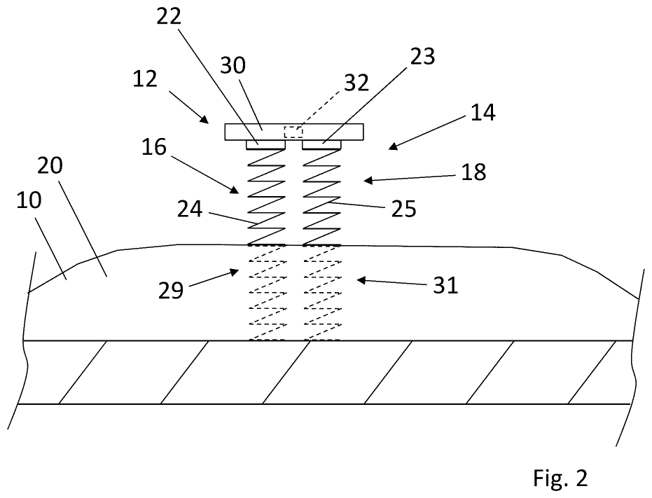

FIG. 2 shows a further sectional view of a body part with a measuring device attached thereto,

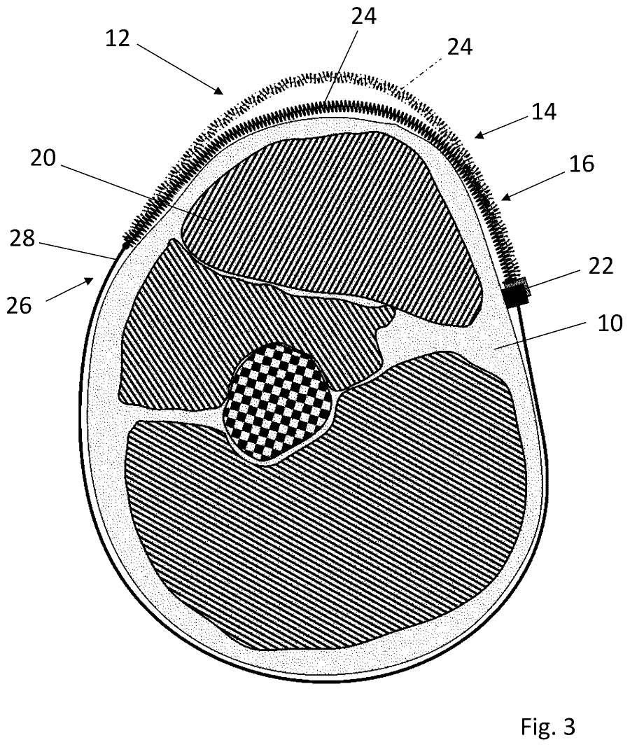

FIG. 3 shows a sectional view of a body part with a measuring device attached thereto according to a further exemplary embodiment, and

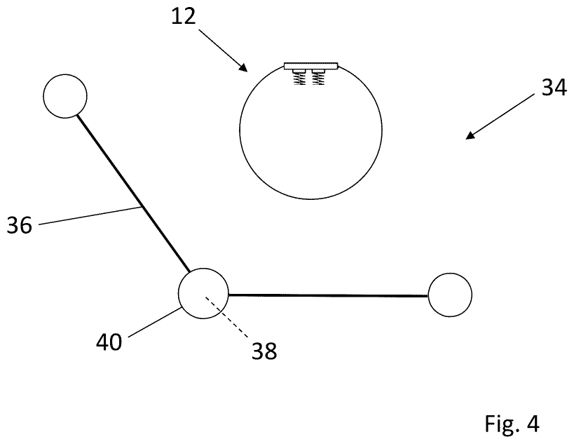

FIG. 4 shows an assistance system comprising a wearable exoskeleton and a wearable measuring device.

FIGS. 1 and 2 show a body part 10 of a person with a wearable measuring device 12 attached to the body part 10. In the present case, the body part 10 is an arm 10 of the person.

FIG. 1 shows a cross-section of the body part 10, in which the sectional plane is aligned perpendicular to the longitudinal extension of the body part 10. FIG. 2 shows a longitudinal section f the body part 10, in which the sectional plane is aligned parallel to the body part 10. It should be noted that FIGS. 1 and 2 are merely schematic representations, so in particular no proportions are to be derived from FIGS. 1 and 2.

The measuring device 12 comprises a measuring apparatus 14 that comprises two measuring units 16 and 18 for monitoring a skeletal muscle 20 of the person. In FIG. 2, the measuring apparatus 14 is shown rotated by 90° compared to FIG. 1, so that both measuring units 16 and 18 can also be seen in FIG. 2. In the following, the measuring unit 16 is referred to as the first measuring unit 16 and the measuring unit 18 as the second measuring unit 18.

In the illustrated exemplary embodiment, the skeletal muscle 20 is the biceps brachii muscle 20 of the arm 10. However, the measuring device 12 can also be used to monitor a different skeletal muscle. In addition, the measuring device 12 can also have a further measuring apparatus in order to simultaneously monitor a plurality of skeletal muscles of the same body part, for example the biceps brachii muscle and the triceps brachii muscle.

The first measuring unit 16 comprises a force sensor 22 and a spring unit 24 associated with the force sensor 22. The second measuring unit 18 also comprises a force sensor 23 and a spring unit 25 associated with the force sensor 23. The lengths of the spring units 24 and 25 are known. The spring units 24 and 25 differ from each other as regards their spring constants. In this case, the spring constant of the spring unit 24 is higher than the spring constant of the spring unit 25. Preferably, the spring constants of the spring units 24 and 25 differ from each other by a factor of at least 1.1. One of the spring constants is then at least 10% higher than the other spring constant.

In the illustrated exemplary embodiment, the spring units 24 and 25 each comprise a coil spring. Such a design of the spring units 24 and 25 is preferred. However, in other exemplary embodiments, the spring units 24 and 25 can also comprise disk springs, gas-pressure springs, beam springs or elastomer springs. In addition, the spring units 24 and 25 can also comprise more than just a single spring element. In particular, the spring units 24 and 25 each comprise a plurality of spring elements that are connected, for example, in series or parallel to one another. In such a design, the spring constant of the spring units 24 and 25 refers to the equivalent spring constant and not to the spring constant of an individual spring element.

The force sensors 22 and 23 are designed to detect a tensioning force of the associated spring unit 24 or 25. In the present case, the force sensors 22 and 23 each comprise a force-dependent electrical resistor for this purpose.

The measuring device 12 also comprises a holding apparatus 26 for attaching the measuring apparatus 14 to the body part 10. In the present exemplary embodiment, the holding apparatus 26 comprises a deformable holding strap 28. In FIG. 2, the holding strap 28 is located outside the sectional plane and is therefore not visible.

In addition to the holding strap 28, the holding apparatus 26 comprises a rigid support structure 30, which in the present case is plate-shaped. The measuring units 16 and 18 are arranged side by side on the support structure 30 and extend in the same direction away from the support structure 30. In the present case, the force sensors 22 and 23 are directly connected to the support structure 30.

The measuring apparatus 14 is attached to the body part 10 by means of the holding apparatus 26 in such a way that the spring units 24 and 25 can be tensioned in each case by a different portion of the skeletal muscle 20. When the skeletal muscle 20 is contracted, the portions of the skeletal muscle 20 consequently exert tensioning forces on the spring units 24 and 25.

A skeletal muscle can be viewed as a grid or array of spring units with the same spring constant. This is indicated in FIG. 2 by dashed springs inside the skeletal muscle 20. The magnitude of the spring constant of the skeletal muscle 20 varies with the degree of contraction of the skeletal muscle. When arranged as intended, the spring units 24 and 25, together with the portion of the skeletal muscle 20 through which they can be tensioned, form a spring arrangement 29 or 31 with an equivalent spring constant. As previously mentioned, the spring units 24 and 25 differ from each other in terms of their spring constant. This means that the spring arrangements 29 and 31 also differ from each other as regards their equivalent spring constant. Because the spring constants of the spring units 24 and 25 differ from one another, the spring units 24 and 25 are each subjected to a different tensioning force when the skeletal muscle 20 is contracted. Specifically, the spring unit 24 having the higher spring constant is subjected to a greater tensioning force than the spring unit 25 having the lower spring constant. Accordingly, the tensioning forces detected by the force sensors 22 and 23 also differ in their magnitude.

The measuring device 12 also comprises an evaluation unit 32, which, in the present case, is integrated into the support structure 30. The evaluation unit 32 is designed to ascertain a spring constant of the skeletal muscle 20 as a function of sensor signals from the force sensors 22 and 23. The spring constant of the skeletal muscle 20 describes a muscle hardness of the skeletal muscle 20. For example, the evaluation unit 32 ascertains the spring constant of the skeletal muscle 20 according to a look-up table, according to a characteristic map, according to a calculation rule or according to a calculation network.

The spring units 24 and 25 each have a first end portion and a second end portion. The end portions are axial portions of the spring units 24 and 25. In the exemplary embodiment shown in the figures, the spring units 24 and 25 are each operatively connected to the force sensors 22 or 23 through the first end portions. The spring units 24 and 25 are operatively connected to the skeletal muscle 20 through the free second end portions.

As can be seen from FIG. 1, the spring units 24 and 25 extend radially in relation to the body part 10. Given this orientation of the spring units 24 and 25, the skeletal muscle 20 exerts tensioning forces on the spring units 24 and 25, which act on the spring units 24 and 25 in the longitudinal extension of the spring units 24 and 25. The spring units 24 and 25 are compressed by the tensioning forces.

In the exemplary embodiment shown in FIG. 1, the spring units 24 and 25 are in direct contact with the user's skin. Alternatively, the measuring device 12 can also be worn over clothing, thereby increasing comfort for the user.

FIG. 3 shows a wearable measuring device 12 according to a further exemplary embodiment. In the measuring device 12 shown in FIG. 3, the spring units 24, 25 extend in the circumferential direction of the body part 10 along the skeletal muscle 20. It should be noted that only the measuring unit 16 is visible in FIG. 3. The other measuring unit 18 is arranged axially offset from the measuring unit 16 in relation to the longitudinal center axis of the body part 10 and is therefore located outside the image plane. The spring unit 24 is shown in simplified form as a coil spring in FIG. 3. However, the spring unit 24 can also be, for example, a textile elastic band. If the monitored skeletal muscle 20 is contracted, the spring units 24, 25 will be subjected to a radially outward-acting tension force and are thereby stretched. This stretched state of the spring unit 24 is indicated by dashed lines in FIG. 3.

FIG. 4 shows a schematic view of an assistance system 34. In the present case, the assistance system 34 comprises, by way of example, the wearable measuring device 12 described above and shown in FIG. 1. In addition, the assistance system 34 comprises a wearable exoskeleton 36, which is shown only in a simplified manner in FIG. 3. The exoskeleton 36 comprises a controllable actuator 38, which in this case is integrated into a joint 40 of the exoskeleton 36.

Furthermore, a control device not shown in the figures is present. The control device is designed to control the actuator 38 as a function of information that can be provided by the measuring device 12. In particular, the control device is also integrated into the joint 40. However, the control device can also be designed externally in relation to the exoskeleton 36. In particular, the control device controls the actuator 38 as a function of the spring constant of the skeletal muscle 20 ascertained by the evaluation unit 32. With such a control of the actuator 38, the exoskeleton 36 can support the function of the skeletal muscle 20, thereby protecting the user's joint and muscle system.

1. A wearable measuring device comprising
a measuring apparatus that comprises at least two measuring units for monitoring a skeletal muscle of a user or a patient, and comprising
a holding apparatus for attaching the measuring apparatus to a body part of the user or the patient having the skeletal muscle, wherein
the measuring units each comprise a force sensor and an associated spring unit, the spring units differing from one another in their spring constant,
the measuring units can be attached to the body part in such a way that the spring units can be tensioned by a particular different portion of the skeletal muscle, and
each force sensor is designed to detect a tensioning force of the correspondingly associated spring unit.

2. The wearable measuring device according to claim 1, further comprising: an evaluation unit configured to ascertain a spring constant of the skeletal muscle as a function of sensor signals from the force sensors.

3. The wearable measuring device according to any of claim 1, wherein at least one of the spring units comprises a metal spring or an elastomer spring.

4. The wearable measuring device according to claim 1, wherein at least one of the spring units is cylindrical, spiral-shaped, plate-shaped, bar-shaped, band-shaped or strand-shaped.

5. The wearable measuring device according to claim 1, wherein at least one of the spring units comprises a gas-pressure spring.

6. The wearable measuring device according to claim 1, wherein at least one of the force sensors comprises at least one force-dependent electrical resistor, at least one force-dependent electrical capacitor, at least one force-dependent electrical inductor or at least one piezoelectric element.

7. The wearable measuring device according to claim 1, wherein the spring units have a first end portion and a second end portion, the associated force sensor being operatively connected to the spring unit through the first end portion, and the spring unit being operatively connectable or operatively connected to the skeletal muscle through the second end portion.

8. The wearable measuring device according to claim 1, wherein the spring units have a first end portion and a second end portion, the associated force sensor being operatively connected to the spring unit through the first end portion, and the spring unit being operatively connectable or operatively connected to the skeletal muscle through the force sensor.

9. The wearable measuring device according to claim 1, wherein the measuring apparatus comprises at least three measuring units.

10. The wearable measuring device according to claim 1, wherein the holding apparatus comprises a holding strap, a holding cuff or a holding bandage.

11. The wearable measuring device according to claim 1, wherein the holding device comprises a support structure or a rigid support structure, and the measuring units being arranged on the support structure or said rigid support structure.

12. The wearable measuring device according to claim 1, wherein the spring units extend radially in relation to the body part when the measuring device is arranged as intended.

13. The wearable measuring device according to any of claim 1, wherein the spring units extend in the circumferential direction of the body part along the skeletal muscle when the measuring device is arranged as intended.

14. An assistance system comprising
a wearable exoskeleton having at least one controllable actuator,
said wearable measuring device according to claim 1, and
a control device that is designed to control the actuator as a function of information that can be provided by the wearable measuring device.

15. A method for determining a muscle hardness of said skeletal muscle of said user or said patient, by means of said wearable measuring device according to claim 1,
at least two spring units that differ from each other in their spring constant,
being attached to a body part of the user or the patient having the skeletal muscle in such a way that the spring units can in each case be tensioned by a different portion of the skeletal muscle,
tensioning forces of the spring units being detected, and
a spring constant of the skeletal muscle being ascertained as muscle hardness as a function of the detected tensioning forces.
